(19) United States
(12) Patent Application Publication
(10) Pub. No.: US 2023/0337911 A1
(43) Pub. Date: Oct. 26, 2023

(54) SYSTEMS AND METHODS TO MEASURE AND IMPROVE EYE MOVEMENTS AND FIXATION

(71) Applicant: Ohio State Innovation Foundation, Columbus, OH (US)

(72) Inventors: Teng Leng Ooi, Columbus, OH (US); Yu-Shiang Jeng, Columbus, OH (US); Zijiang He, Columbus, OH (US)

(21) Appl. No.: 18/307,512

(22) Filed: Apr. 26, 2023

Related U.S. Application Data
(60) Provisional application No. 63/334,792, filed on Apr. 26, 2022.

(51) Int. Cl. A61B 3/113; G02B 27/01; A61B 3/08; A61B 3/00

(52) U.S. Cl. CPC A61B 3/113; G02B 27/0172; A61B 3/08; A61B 3/005; G02B 2027/014; G02B 27/0189

(57) ABSTRACT

A gaze-tracking method and system are disclosed that employs gaze rays generated from measured/tracked eye movements and fixation for interaction with objects and environment within a three-dimensional computing environment. The gaze ray is tracked to provide measures of ocular control, fovea control, and/or associated neural pathways in which the measures can be then used for one or more physiological assessments of the user, including, for example, eye dominance determination, vestibular assessment, visual fixation assessment, optokinetic assessment, smooth pursuit assessment, nystagmus quick phase assessment, saccades assessment, and vergence assessment.

### RELATED APPLICATION

This application claims priority to, and the benefit of, U.S. Provisional Patent Application No. 63/334,792, filed Apr. 26, 2022, entitled “Systems and Methods to Measure and Improve Eye Movements and Fixation,” which is hereby incorporated by reference herein in its entirety.

### BACKGROUND

There is a benefit in being able to measure and understand eye movements and fixation behaviors as it relates to the performance of daily human activities as well as for athletic training and vocational purposes. There is also a benefit in assessing the control of eye movements and fixation in the role of human functional and social interaction.

Eye movements and fixation generally involve ocular muscles, the control of the fovea, and the associated neural pathways for image processing and motor controls. The fovea is a small, specialized area with the highest concentration of cone photoreceptor cells in the retina. Movement of the eyeball in the direction of interest, along with the adjustment of the retina allows the eye to fixate on objects in the visual scene, judge their distances, track their movements, and see fine details, allowing a person to perceive and scrutinize their visual environment. Highly developed fine control of eye movements and stable eye fixation can be developed through practice through sports (e.g., from archery to water polo) and vocational tasks such as surgeries. Gaze and fixation behavior of a person also attributed to human behavior are also observed and assessed, sometimes explicitly and instinctively, by another person to evaluate their intention and focus during normal conversation and interaction.

Established eye-tracking instruments and tests do not provide assessments of real-time natural eye movement behaviors in 3D space. While existing eye-tracking instruments can measure the subject's eye movement behaviors in response to various stimuli presented on a flat 2D computer monitor (along the frontal plane), the eye movement data collected are often analyzed independently of the instruments in an offline manner. The assessment also provides the averaged eye movement characteristics of the subject, such as eye movement velocity, amplitude, and accuracy.

There is a benefit to measuring and improving eye movements and fixation in the clinical diagnosis and therapy of eye movement and fixation problems.

### SUMMARY

A gaze-tracking method and system are disclosed that employs gaze rays generated from measured/tracked eye movements and fixation for interaction with objects and environment within a three-dimensional computing environment. The gaze ray is tracked to provide measures of ocular control, fovea control, and/or associated neural pathways in which the measures can be then used for one or more physiological assessments of the user, including, for example, eye dominance determination, vestibular assessment, visual fixation assessment, optokinetic assessment, smooth pursuit assessment, nystagmus quick phase assessment, saccades assessment, and vergence assessment.

The gaze control can be used as a part of hand-free user interface input for interactions with a virtual or augmented three-dimensional computing environment. In some embodiments, the gaze-tracking method and system are used with a head-mounted display virtual reality system that can provide measured eye movement and fixation information from a user. In other embodiments, the gaze-tracking method and system are used with an augmented-reality system that can provide measured eye movement and fixation information from a user.

The gaze-tracking method and system can provide concurrent/simultaneous visualization of a user's eye movement behavior, in addition to metrics such as velocity, amplitude, and accuracy, in real-time to both (i) the user and (ii) a clinician, technician, or examiner who is evaluating the user's eye movement behavior for an assessment of ocular control, fovea control, and/or associated neural pathways.

In an aspect, a system is disclosed comprising a virtual-reality headset or augmented-reality system configured to measure eye-associated positions and gaze-associated direction; and an analysis system having a processor and a memory having instructions stored thereon, wherein execution of the instructions by the processor cause the processor to generate a rendered scene and/or object in the virtual-reality headset or augmented-reality system; receive the measured eye associated positions and measured gaze-associated directions of a user viewing the rendered scene and/or object; determine a gaze control point from the measured eye associated positions and measured gaze-associated directions, wherein the gaze control point is a binocular convergence in the rendered scene and/or on the object using the measured gaze associated directions from both eyes to reflect a combined eye movement pattern.

In some embodiments, execution of the instructions by the processor further causes the processor to generate a rendered gaze ray associated with the binocular convergence.

In some embodiments, execution of the instructions by the processor further causes the processor to determine one or more statistical parameters, or associated values, from the determined gaze control point.

In some embodiments, the determined one or more statistical parameters includes at least one of (i) a variance measure of the gaze control point, (ii) a mean location of the gaze control point, (iii) a latency measure of the gaze control point, (iv) a change measure in the variance of the gaze control point over time, (v) a change measure in the latency of the gaze control point over time, (vi) an instantaneous location of the gaze control point, (vii) an instantaneous velocity measure of the gaze control point, and (viii) an instantaneous acceleration measure of the gaze control point.

In some embodiments, the determined gaze control point, or statistics derived therefrom, are subsequently employed to assess at least one of eye dominance measure, vestibular measure, visual fixation measure, optokinetic measure, smooth pursuit measure, nystagmus quick phase measure, saccades measure, vergence measure, or a combination thereof.

In some embodiments, the determined gaze control point, or statistics derived therefrom, are subsequently employed in a therapy to address an eye-tracking problem or a disease.

In another aspect, a system is disclosed comprising an analysis system having a processor and a memory having instructions stored thereon, wherein execution of the instructions by the processor causes the processor to: generate a rendered scene and/or object in the virtual-reality headset or augmented-reality system; receive from a VR or AR system (e.g., VR headset) measured eye-associated positions and measured gaze associated directions of a user viewing the rendered scene and/or object; determine a gaze control point from the measured eye associated positions and measured gaze-associated directions, wherein the gaze control point is a binocular convergence in the rendered scene and/or on the object using the measured gaze associated directions from both eyes to reflect a combined eye movement pattern.

In another aspect, a virtual-reality headset is disclosed comprising a processor; and a memory having instructions stored thereon, wherein execution of the instructions by the processor causes the processor to: display a rendered scene and/or object; measure eye-associated positions of a user wearing the virtual-reality headset; measure gaze associated directions of the user; and determine a gaze control point from the measured eye associated positions and measured gaze-associated directions, wherein the gaze control point is a binocular convergence in the rendered scene and/or on the object using the measured gaze associated directions from both eyes to reflect a combined eye movement pattern. The determined gaze control point, or statistics derived therefrom, may be subsequently employed to assess at least one of: eye dominance measure, vestibular measure, visual fixation measure, optokinetic measure, smooth pursuit measure, nystagmus quick phase measure, saccades measure, vergence measure, or a combination thereof. The determined gaze control point, or statistics derived therefrom, may be subsequently employed in a therapy to address an eye-tracking problem or a disease.

The virtual-reality headset may include any of the features of any of the above-discussed systems.

In another aspect, various methods are disclosed with respect to the operations of the above-discussed systems.

In another aspect, a non-transitory computer-readable medium is disclosed having instructions stored thereon, wherein execution of the instructions by a processor causes the processor to: generate a rendered scene and/or object in the virtual-reality headset or augmented-reality system; receive, from a virtual-reality headset or augmented-reality system, measured eye associated positions and measured gaze associated directions of a user viewing the rendered scene and/or object; determine a gaze control point from the measured eye associated positions and measured gaze-associated directions, wherein the gaze control point is a binocular convergence in the rendered scene and/or on the object using the measured gaze associated directions from both eyes to reflect a combined eye movement pattern. The determined gaze control point, or statistics derived therefrom, may be subsequently employed to assess at least one of eye dominance measure, vestibular measure, visual fixation measure, optokinetic measure, smooth pursuit measure, nystagmus quick phase measure, saccades measure, vergence measure, or a combination thereof. The determined gaze control point, or statistics derived therefrom, may be subsequently employed in a therapy to address an eye-tracking problem or a disease.

The non-transitory computer-readable medium may include instructions that can execute the features of any of the above-discussed systems and methods.

### BRIEF DESCRIPTION OF THE FIGURES

The accompanying drawings, which are incorporated in and constitute a part of this specification, illustrate embodiments and together with the description, serve to explain the principles of the methods and systems.

Embodiments of the present invention may be better understood from the following detailed description when read in conjunction with the accompanying drawings. Such embodiments, which are for illustrative purposes only, depict novel and non-obvious aspects of the invention. The drawings include the following figures:

FIGS. 1A and 1B each show an example of a gaze-tracking system configured with gaze ray control or assessment generated from measured/tracked eye movements and fixation of a user while the user interacts with objects and environment within a three-dimensional computing environment or augmented reality environment in accordance with an example embodiment.

FIGS. 2A and 2B each show a system that provides gaze control points and derived statistics can be used as a part of hand-free user interface input for interactions with the virtual three-dimensional computing environment to provide measures of ocular control, fovea control, and/or associated neural pathways in which the measures can be then used for therapy of eye-tracking problems or issues as well as a neurological disease in accordance with an example embodiment.

FIGS. 3A and 3B each show plots of data acquired from the gaze control point for the left eye and the right eye. Each plot (302, 304) shows the x, y, z positions (306, 308, 310) of the gaze control point.

FIG. 4A shows a method for gaze-collision control that calculates a point of binocular convergence in a 3D interactive space using the gaze rays from both eyes to reflect the combined eye movement patterns in 3D space in accordance with an example embodiment.

FIGS. 4B and 4C provide an example pseudocode and example executable code for the operation provided in FIG. 4A in accordance with an example embodiment.

FIGS. 5A and 5B show example hardware configurations for the AR/VR systems that can be employed with the exemplary gaze-tracking method and system in accordance with an example embodiment.

FIGS. 6A, 6B, 6C, 6D, 6E, and 6F show example applications for gaze-associated diagnostics and/or training in accordance with an example embodiment.

FIGS. 7A, 7B, and 7C show example output analysis visualization for the visuomotor function analysis performed in the diagnostic or training exercise described herein in accordance with an example embodiment.

### DETAILED SPECIFICATION

Some references, which may include various patents, patent applications, and publications, are cited in a reference list and discussed in the disclosure provided herein. The citation and/or discussion of such references is provided merely to clarify the description of the present disclosure and is not an admission that any such reference is “prior art” to any aspects of the present disclosure described herein. In terms of notation, “[n]” corresponds to the nth reference in the list. All references cited and discussed in this specification are incorporated herein by reference in their entireties and to the same extent as if each reference was individually incorporated by reference.

Some references, which may include various patents, patent applications, and publications, are cited in a reference list and discussed in the disclosure provided herein. The citation and/or discussion of such references is provided merely to clarify the description of the disclosed technology and is not an admission that any such reference is “prior art” to any aspects of the disclosed technology described herein. In terms of notation, “[n]” corresponds to the nth reference in the list.

Vision is arguably the most developed sense humans are endowed with. The retina of each eye has a small, specialized area with the highest concentration of cone photoreceptor cells known as the fovea, which gives us the ability to see fine details. We constantly depend on our foveae to scrutinize our visual environment by moving the fovea of each eye (i.e., eye movement) in the direction of an object of interest (i.e., to fixate). Athletes in various sports ranging from archery to water polo depend on their highly developed ability to control their eye movements and fixation, through practice, to excel over their competitors. Skilled surgeons also depend on fine control of eye movements and stable eye fixation to perform delicate surgeries. Besides specialized populations, every day, we rely on our foveae to scrutinize the visual environment and to fixate on objects in the visual scene to judge their distances and track their movements. We look a person in the eyes when speaking with them. Undoubtedly, the control of eye movements and fixation is vital for functional and social interaction. Hence, it is important to measure and understand eye movements and fixation behaviors for the performance of activities of daily living as well as for athletic and vocational purposes.

Most existing eye-tracking instruments measure the subject's eye movement behaviors in response to various stimuli presented on a flat 2D computer monitor (along the frontal plane). The eye movement data collected are then analyzed independently, usually offline, to reveal the averaged eye movement characteristics of the subject. However, while the examiner gets to appreciate the averaged data in detail after the data are analyzed, such as the velocity, amplitude, and accuracy of the eye movements, neither the examiner nor the subject gets to visualize the eye movement behaviors simultaneously in real-time to receive instantaneous feedback, which is crucial for modifying behaviors (diagnostic and therapeutic).

Furthermore, we live in a 3D environment, and our eyes naturally look at objects both along with the frontal and sagittal (depth) planes. Yet, we know very little about how our eyes behave in 3D space because most existing technology measures eye movements in 2D. To learn about eye movements in 3D, we need to measure each eye's movements independently. The difference in movements between the two eyes provides crucial information about their behaviors in 3D.

However, while existing VR software protocols may include eye-tracking and fixation capabilities, they are being employed for the improvement of the computing environment, e.g., using Foveated-based rendering [1], and not for clinical diagnostics or therapy.

Because the absolute magnitude of difference between the movements of the two eyes is in the order of minutes of arc, eye tracking instruments with better spatial and temporal resolutions are beneficial to provide adequate visualization of the resultant 3D eye movement data. These 3D eye movement data can also inform clinicians whether a patient with neurological deficits (e.g., from neural degeneration or trauma) suffers from poor movement coordination between the two eyes, which can degrade depth perception.

The exemplary system can measure eye movements and fixation behaviors in the 3D visual environment and can assist researchers and clinicians in understanding 2D behaviors to 3D in relation to the different neural circuitries. The exemplary system can be employed to track eye movements and fixation behaviors in 3D space while wearing the headset. The exemplary system can measure eye and head (gaze) movement behaviors in 3D scenes, as well as provide visualization of the gaze behaviors simultaneously (online). The exemplary system can be implemented in VR headsets to add functions to native hardware and firmware eye-tracking and fixation functions. The exemplary system can provide gaze ray-based controls that allow the subjects to visualize where their eyes are moving and fixating in real-time, thus providing them with instantaneous feedback about their ocular-motor behaviors.

### Example System #1

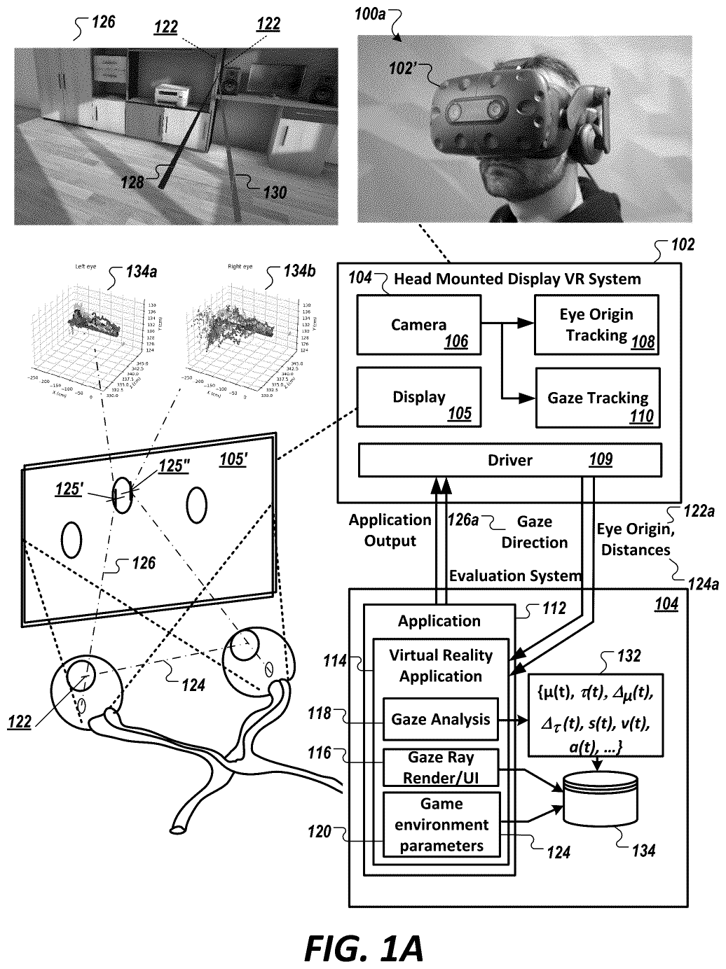

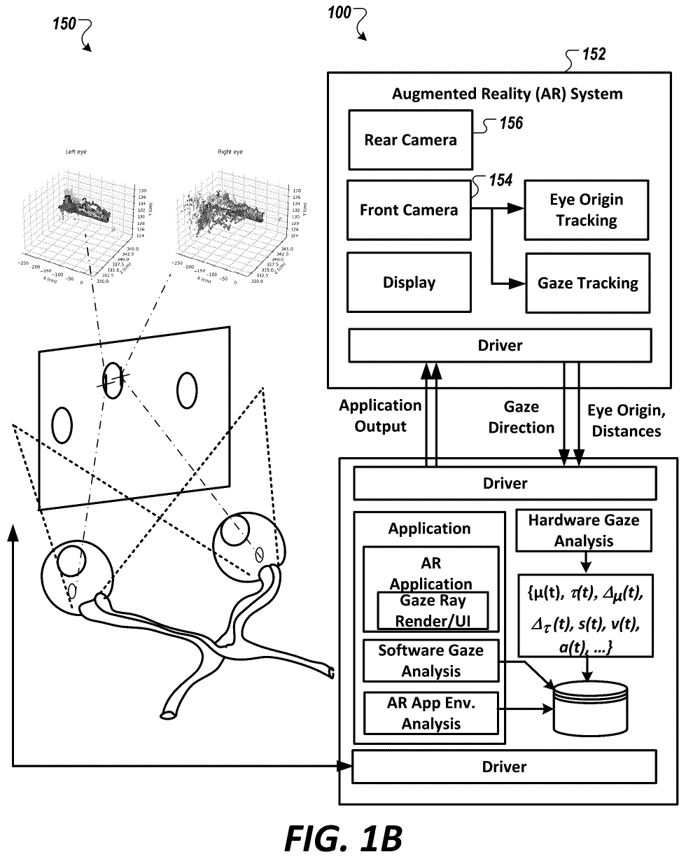

FIG. 1A shows an example of a gaze-tracking system 100 (shown as 100a) configured with gaze ray control or assessment generated from measured/tracked eye movements and fixation of a user while the user interacts with objects and environment within a three-dimensional computing environment. In the example shown in FIG. 1A, the system 100a includes a head-mounted display VR system 102 (an example shown as 102′) that operates with an evaluation system 104.

In the example shown in FIG. 1A, the head-mounted display VR system 102 includes a display 105 and camera system 106 that operates with an eye origin tracking module 108 and gaze tracking module 110.

The head-mounted display VR system 102 includes a driver interface 109 that can provide outputs 111 from the eye origin tracking and gaze tracking modules (108, 110), such as eye origin measurements 122, the distances between the two eyes 124, and measured gaze directions 126 (also referred to as gaze rays 126). The left and right eye origin parameters 122a (or gaze origin parameters 122a) and the left and right eye gaze direction parameters 126a can each be provided, e.g., as a vector comprising 3 floating point numbers, or other data representation, to determine the target 125 (shown as 125′ and 125″). An example of a head-mounted display VR system (e.g., 102′) is the Vive Pro Eye VR headset (manufactured by HTC Technologies).

The evaluation system 104 includes an evaluation application 112 (shown as “Application” 112) that includes a virtual reality application 114 having a gaze ray render/UI module 116, a gaze analysis module 118, and a game environment parameter module 120. The evaluation system 104 may be implemented in or executed with a gaming engine 119 (not shown) for the head-mounted display VR system 102. An example of a gaming engine is the Unity game engine (manufactured by Unity Technologies). Unity game engine includes functions to provide users the ability to create games and interactive application experiences in both 2D and 3D. The engine can be programmed through a primary scripting API in C#, e.g., using Mono.

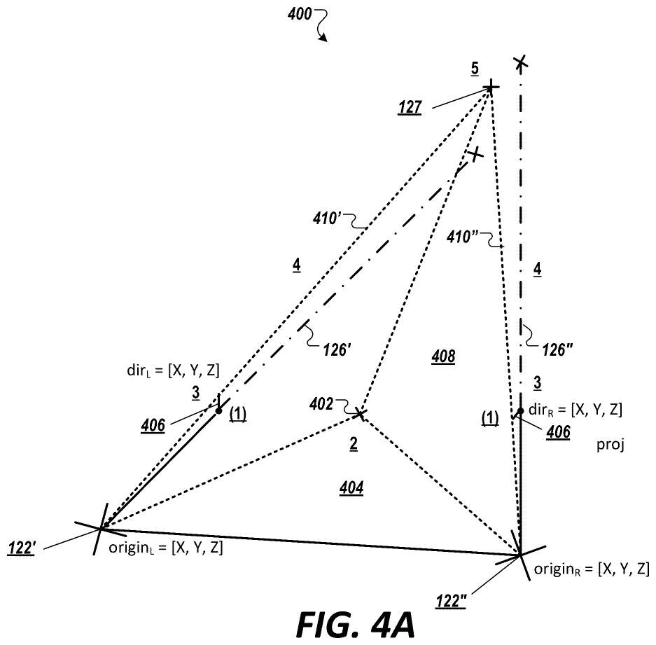

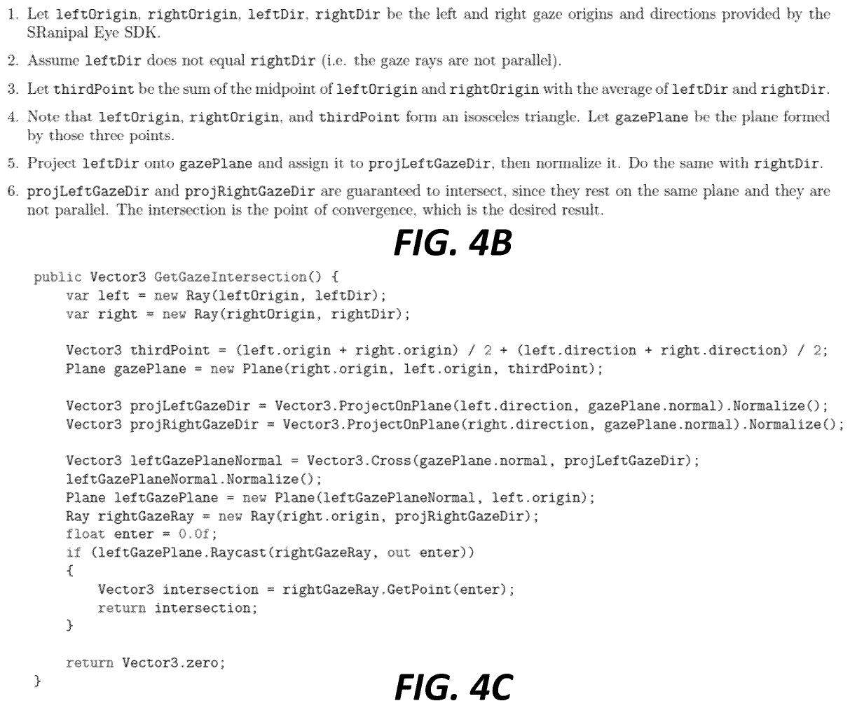

The gaze ray render/UI module 116 is configured to access the drivers 109 of the head-mounted display VR system 102 and use the left and right eye origin parameters (e.g., 122a) (or gaze origin parameters) and the left and right eye gaze direction parameters (e.g., 126a) to execute gaze-collision control that calculates a point of binocular convergence 127 (not shown—see FIG. 4A) (referred to as the gaze control point 127) in the 3D interactive space using the gaze rays 126 from both eyes to reflect the combined eye movement patterns in 3D space. The gaze rays 126 may be rendered into the 3D virtual environment, e.g., on display 105, as part of the user interface of the VR system 102. In the example shown in FIG. 1A, the gaze ray for the left eye is rendered as a blue line 128, and the gaze ray for the right eye is rendered as a red line 130. In alternative embodiments, the rendered gaze ray (e.g., 128, 130) may be assigned a color based on the dominance of the eye, e.g., red assigned to the dominant eye and blue assigned to the non-dominant eye.

The gaze analysis module 118 can log and store the logged data with respect to a test sequence, including the left and right eye origin parameters (e.g., 122a) (or gaze origin parameters) and the left and right eye gaze direction parameters provided by the head-mounted display VR system (102). The gaze analysis module 118 can determine a set of statistical parameters 132 from the gaze control point 127 that can be used for diagnostics (e.g., clinical diagnostics) or for training assessment (e.g., within fixation training), as described herein. The statistical parameters 132 may include (i) a variance μ(t) of the gaze control point, (ii) a mean location of the gaze control point, (iii) a latency τ(t) of the gaze control point, (iv) a change in the variance Δ_{μ}(t) over time of the gaze control point, (v) a change in the variance Δ_{τ}(t) over time of the gaze control point, (vi) a location s(t) of the gaze control point, (vii) a velocity measure v(t) of the gaze control point, and (viii) an acceleration a(t) of the gaze control point, among others. Plots 134a, 134b show examples of the measured gaze control point.

Gaze control data store. The system may include a data store 134 configured to receive, store, and/or log the determined outputs from the gaze analysis module 118, the gaze ray render/UI module 116, as well as game environment parameters 120. The game environment parameters 120 can include metadata and properties data of the 3D object or 3D environment that is interacted with by the gaze control point 127 during a test sequence. The metadata can include the state of the 3D object or 3D environment object, e.g., the duration of time that the gaze control point is on the 3D object or 3D environment object, the frequency of interaction, among others. The properties data include the information of the object, e.g., name, geometry, type, labels, color, etc.

While shown as separate modules or systems, it should be appreciated that the analysis system (e.g., 104) of FIG. 1A or 1B may be integrated into a virtual reality system (e.g., the VR head-mounted system (e.g., 102) of FIG. 1A).

### Example Gaze-Associated Measurement

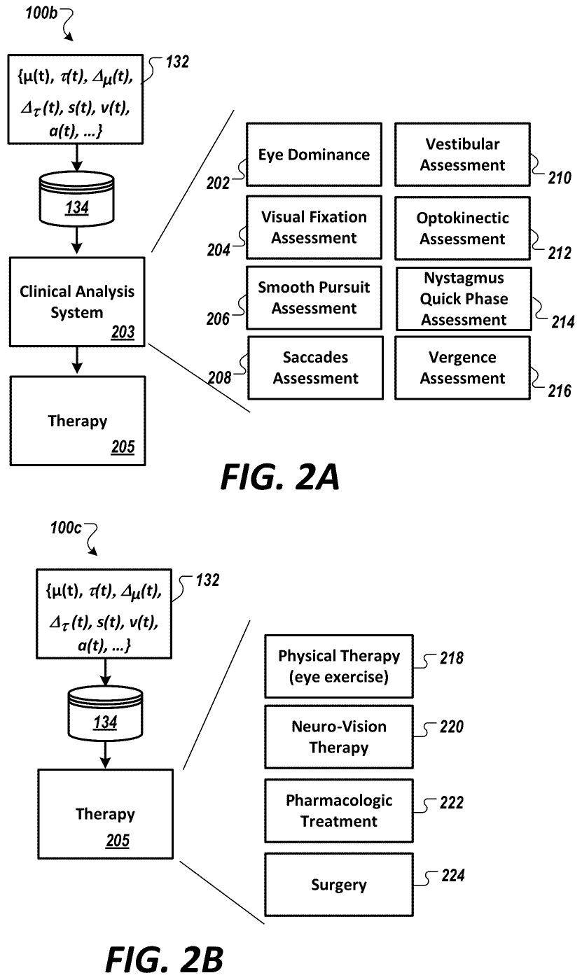

The gaze control point 127 can be used as a part of hand-free user interface input, e.g., for a system 100 (shown as 100b), for interactions with the virtual three-dimensional computing environment to provide measures (e.g., 132) of ocular control, fovea control, and/or associated neural pathways, e.g., to a clinical analysis system 203, in which the measures can be then used as one or more physiological assessment or therapy 205 of the user, including, for example, eye dominance determination, vestibular assessment, visual fixation assessment, optokinetic assessment, smooth pursuit assessment, nystagmus quick phase assessment, saccades assessment, and vergence assessment (see FIG. 2A).

The eye dominance assessment analysis (and associated module) (202) determines, using one of the statistical parameters (variance, change in variance, latency, change in latency, location, velocity, acceleration), and outputs a score or value that indicates the dominance of the right eye, the left eye or the indication of the right eye, left eye, or neither as being dominant. It has been observed that the dominant eye would exhibit less variance as compared to the non-dominant eye.

The visual fixation assessment analysis (and associated module) (204) determines, using one of the statistical parameters (variance, change in variance, latency, change in latency, location, velocity, acceleration, among other metrics described herein), and outputs a score or value that is associated with the ability of the subject to maintain gaze on a stationary 3D object steady on the fovea by minimizing ocular drifts.

The smooth pursuit assessment analysis (and associated module) (206) determines, using one of the statistical parameters (variance, change in variance, latency, change in latency, location, velocity, acceleration, among other metrics described herein), and outputs a score or value that is associated with the ability of the subject to maintain gaze on a small moving 3D object steady on the fovea.

The saccades assessment analysis (and associated module) (208) determines, using one of the statistical parameters (variance, change in variance, latency, change in latency, location, velocity, acceleration, among other metrics described herein), and outputs a score or value that is associated with the ability of the subject to bring a 3D object of interest onto the fovea.

The vestibular assessment analysis (and associated module) (210) determines, using one of the statistical parameters (variance, change in variance, latency, change in latency, location, velocity, acceleration, among other metrics described herein), and outputs a score or value that is associated with the ability of the subject to maintain the image of the 3D world on the retina during a brief head rotation or translation of the subject's head.

The optokinetic assessment analysis (and associated module) (212) determines, using one of the statistical parameters (variance, change in variance, latency, change in latency, location, velocity, acceleration, among other metrics described herein), and outputs a score or value that is associated with the ability of the subject to maintain the scene of the 3D world on the retina during sustained head rotation and/or motion of the visual field (e.g., when viewing scenes from the window of a moving train) for gaze stabilization.

The nystagmus quick phase assessment analysis (and associated module) (214) determines, using one of the statistical parameters (variance, change in variance, latency, change in latency, location, velocity, acceleration, among other metrics described herein), and outputs a score or value that is associated with the ability of the subject to reset the eye during prolonged rotation and direct gaze towards an oncoming visual scene.

The vergence assessment analysis (and associated module) (216) determines, using one of the statistical parameters (variance, change in variance, latency, change in latency, location, velocity, acceleration, among other metrics described herein), and outputs a score or value that is associated with the ability of the subject to move the eyes in opposite directions so that images of a single object are placed or held simultaneously on the fovea of each eye.

The various assessment analyses and modules may employ machine learning or artificial intelligence algorithms as well as classical control analysis and modeling.

### Example Gaze Control Data

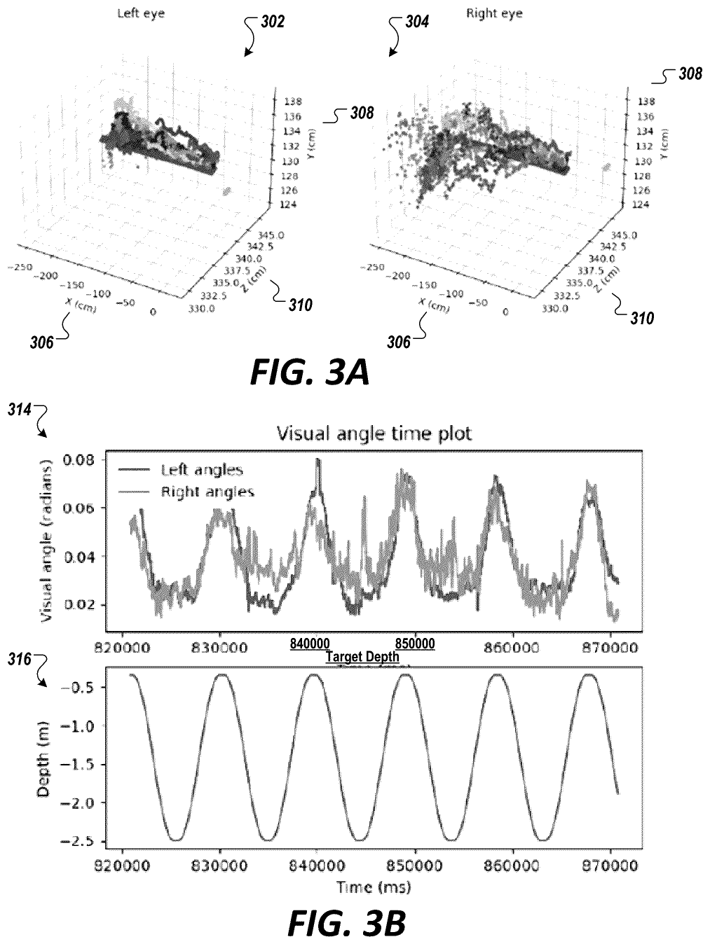

FIG. 1A (reproduced below as FIG. 3A) shows two plots (302, 304) of an example data set acquired of the gaze control point 127 for the left eye and the right eye. Each plot (302, 304) shows the x, y, z positions (306, 308, 310) of the gaze control point.

FIG. 3B shows additional example plots (314, 316) of the gaze control point 127. In FIG. 3B, the visual angle time is shown. The x-axis shows the time (in milliseconds), and the y-axis shows the visual angle (in radians) and depth (in meters).

FIGS. 3A and 3B show data acquired from a subject that is asked to focus on a target moving away and toward the eyes within time window of 50 seconds. The target, in this instance, was a red ball. The eye-tracking movements can be replayed afterward. During the data acquisition, the system is configured to either render the gaze rays for the user or to omit the gaze rays. As shown in FIGS. 3A and 3B, the eye tracking movements are recorded and can then be subsequently analyzed (e.g., per the plot) to predict eye dominance.

In some embodiments, the gaze-tracking method and system is used with a head-mounted display virtual reality system (e.g., FIG. 1A) that can provide measured eye movement and fixation information from a user. In other embodiments, the gaze-tracking method and system is used with an augmented-reality system (e.g., FIG. 1B) that can provide measured eye movement and fixation information from a user.

The gaze-tracking method and system can provide concurrent/simultaneous visualization of a user's eye movement behavior, in addition to metrics such as velocity, amplitude, and accuracy, in real-time to both (i) the user and (ii) a clinician, technician, or examiner who is evaluating the user's eye movement behavior for an assessment of ocular control, fovea control, and/or associated neural pathways.

The gaze-tracking method and system can be used for disease assessments such as Parkinson's, Alzheimer's, optometric, and various ophthalmological issues. As noted, it has been observed that the non-dominant eye has a higher variance in gaze fixation.

In the context of a video game, the system can adjust the level of difficulty of the game while monitoring eye variance. The system can have the subject play a game to the most difficult or taxing level for the subject and assess the subject's gaze control at that level, e.g., to assess impairments or the presence or non-presence of dominance effects.

### Example Clinical Assessment

FIG. 2B shows a system 100 (shown as 100c) that provides gaze control points and derived statistics 132 can be used as a part of hand-free user interface input for interactions with the virtual three-dimensional computing environment to provide measures of ocular control, fovea control, and/or associated neural pathways in which the measures can be then used for therapy of eye-tracking problems or issues as well as a neurological disease.

The vision therapy (eye exercise) (and associated module) (218) determines, using one of the statistical parameters (variance, change in variance, latency, change in latency, location, velocity, acceleration, among other metrics described herein), and outputs a score or value that is used to adjust a physical therapy activity for the subject.

As one example, in the context of a video game, the vision therapy system can adjust the level of difficulty of the game while monitoring eye variance. The system can have the subject play a game to the most difficult or a fairly taxing level for the subject and assess the subject's gaze control at that level, e.g., to assess impairments or the presence or non-presence of dominance effects. The system can use the game to train the subject's ability of control fixation, e.g., to increase or decrease dominance (e.g., change the variance of the gaze control point).

The neuro-vision therapy (and associated module) (220) determines, using one of the statistical parameters (variance, change in variance, latency, change in latency, location, velocity, acceleration, among other metrics described herein), and outputs a score or value that is used to adjust a physical therapy activity for the subject that involve hand and eye coordination.

The pharmacological therapy (and associated module) (222) determines, using one of the statistical parameters (variance, change in variance, latency, change in latency, location, velocity, acceleration, among other metrics described herein), and outputs a score or value that is used to adjust a pharmacological therapy activity for the subject to treat a neurological issue, an optometric issue, an ophthalmological issue, or a combination thereof.

The surgery therapy (and associated module) (224) determines, using one of the statistical parameters (variance, change in variance, latency, change in latency, location, velocity, acceleration, among other metrics described herein), and outputs a score or value that is used to inform surgery on the subject to treat a neurological issue, an optometric issue, an ophthalmological issue, or a combination thereof.

### Example Method to Determine Gaze Control Point

FIG. 4A shows a method 400 for gaze-collision control that calculates a point of binocular convergence 127 (i.e., gaze control point) in the 3D interactive space using the gaze rays 126 (shown as 126′ and 126″) from both eyes to reflect the combined eye movement patterns in 3D space.

Outputs of eye-tracking measurements generally include two sets of vectors (e.g., corresponding to 126′ and 126″) that are each an average of the gaze ray from a given eye. Combining the average value of these two vectors assume that the two eyes are equally capable of fixating on an object of interest. To more accurately determine the combined eye movements in 3D space while maintaining the independence of each eye's characteristic pattern of movement, the exemplary method and system can calculate a point 127 of binocular convergence (i.e., gaze control point) in the 3D interactive space using the gaze rays (126′, 126″) from both eyes to reflect the combined eye movement patterns in 3D space. In some embodiments, only measurements of one eye can be used.

In the example shown in FIG. 4A, the system receives the measured left gaze origin 122′, 122″ (shown as “origin_{L}” 122′ and “origin_{R}” 122″) and the measured gaze ray 126′, 126″ for the left and right eyes. The system then calculates a center point 402 as the sum of the midpoint of the measured left gaze origin 122′, 122″ and the average of the measured gaze ray 126′, 126″. The system then forms a triangle 404 from the center point 402 and the left gaze origin 122′, 122″. The gaze ray 126′, 126″ are then projected (406) onto a plane 408 located on the triangle 404 and normalized. The resulting normalized projected lines 410′, 410″ are ensured to have the point of convergence 127 since they are on the same plane and not parallel to one another. FIGS. 4B and 4C provide an example implementation of the operation. FIG. 4B provides a pseudocode for the operation provided in FIG. 4A, and FIG. 4C provides an example executable code of the pseudocode instructions of FIG. 4B.

### Example #2: Example Augmented Reality System

FIG. 1B shows an example of a gaze-tracking system 100 (shown as 100d) configured with gaze ray control or assessment generated from measured/tracked eye movements and fixation of a user while the user interacts with objects and environment within an augmented reality (AR) computing environment 152 (shown as “Augmented Reality (AR) System” 152).

The AR system 152 includes a front camera 154 and a rear camera 156 to generate 3D objects and render such objects into the images or video frames captured by the front camera 154. In contrast, the virtual reality headset 102 may include eye-tracking cameras 106. In some embodiments, the virtual reality/augmented reality headset (e.g., 102, 152) may include multiple cameras to generate 2D or 3D objects and render such objects into the images or video frames captured by its front camera 154 (or camera 106).

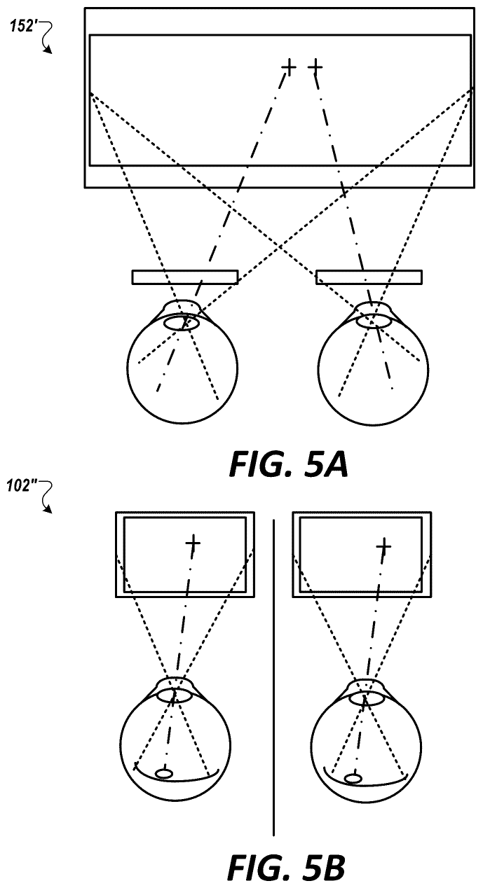

FIGS. 5A and 5B show example hardware configurations for the AR/VR systems (e.g., 102, 152, shown as 102″ and 152′) that can be employed with the exemplary gaze-tracking method and system.

### Example Gaze-Associated Diagnostic and/or Training Applications

As noted above, the gaze analysis module (e.g., 118) can determine a set of statistical parameters (e.g., 132) from the gaze control point (e.g., 127) that can be used for diagnostics (e.g., clinical diagnostics) or for training assessment (e.g., diagnostics within a training application) in a 2D or 3D application.

In each of the applications, the system presents one or more objects that require the user to fixate on a target presented in the environment. During the fixation, the system can determine one or more of (i) a variance μ(t) of the gaze control point, (ii) a mean location of the gaze control point, (iii) a latency τ(t) of the gaze control point, (iv) a change in the variance 41(t) over time of the gaze control point, (v) a change in the variance Δ_{τ}(t) over time of the gaze control point, (vi) a location s(t) of the gaze control point, (vii) a velocity measure v(t) of the gaze control point, and (viii) an acceleration a(t) of the gaze control point, and stored those parameters for clinical diagnostics or for diagnostics of the training exercise. Different classes of applications (modeled as video games) are contemplated and developed.

Table 1 shows an example list of applications.

TABLE 1

| | | Example Visuomotor |
|---|---|---|
| | | Function Diagnostic |
| | Application Description | of User |
| Type 1 | Single stationary object is presented | Visual search perception |
| | for the user to fixate | Eye fixation accuracy |
| | | Eye fixation stability |
| Type 2 | Multiple stationary objects are | Visual search perception |
| | presented for the user to select and | Eye fixation accuracy |
| | fixate | Eye fixation stability |
| Type 3 | Single or multiple moving objects are | Visual search perception |
| | presented for the user to fixate | Eye Pursuit |
| | | Eye Convergence |
| | | Visual Attention |
| Type 4 | Single or multiple moving objects are | Visual search perception |
| | presented for the user to fixate for a | Eye Pursuit |
| | period of time to select and then | Eye Convergence |
| | perform a task with respect to the | Visual Attention |
| | selected object | |
| Type 5 | Multiple hidden stationary objects are | Visual search perception |
| | concurrently presented for the user to | Eye fixation accuracy |
| | search for within the scene and then | Eye fixation stability |
| | fixate. The user may be able to move | Eye saccadic search |
| | within the scene to search for the | pattern |
| | object. | |

In Table 1, the system can present a single active object to the user to fixate (type “1”). The active object may be a stationary animated object (e.g., an animated balloon, an animated target, or any animated object). The animated object may be rotating, oscillating, or varying in some manner in a stationary location.

In a similar type of application (type “2”), the system can present multiple static targets of different types in the environment (e.g., objects in the background of the game) for the user to fixate. The static target is fixed spatially and has no rotation, oscillation, or movement.

In another type of application (type “3”), the system can present one or more actively moving objects that are animated and moving in spatial locations within the environment. A single object can be presented for the user to fixate, or multiple objects can be presented for the user to select among the ground and then fixate thereupon.

In another type of application (type “4”), the system can present one or more active objects for the user to perform an action or activity. For example, the user may first fixate upon one active or static object in the scene for a pre-defined period, then move the object by moving the gaze to another object.

In another type of application (type “5”), the system can present one or more inconspicuous objects within the scene for the user to have to search within the scene and identify and then fixate. The system may allow the user to move within the scene to search for the object.

Indeed, for training applications, the number of objects or targets can be varied or specified. The object can be presented in the same location all the time or can be randomized (so the user would have to identify and search for them). In addition, to adjust the difficulty of the task and/or to assess at different levels of difficulty, the system can vary the contrast and/or luminance of the object or target. The system may also present the object or target in different sizes, e.g., in diminishing size. For purely diagnostic applications, the system would present a standard target or object so it could be normalized for the population.

Other visuomotor functions may be trained or assessed, including those described in relation to FIGS. 2A and 2B.

Diagnostic Features.

Within the application, the system can provide visual cues to assist with the training application or the diagnostic. The system can also be configured or selected to employ different feedback for the diagnostics and/or the training exercise. Table 2 shows a summary of the different diagnostic feature configurations.

TABLE 2

| Diagnostics | Training/Therapeutics |
|---|---|
| Gaze rays off | Gaze rays on for biofeedback |
| Individual eye tested | Gaze rays off to remove feedback |
| Both eyes tested together | Individual eye training |
| Standard testing parameters | Both eye trained together |
| | Different levels of game difficulties |
| | Game parameters customizable |

Per Table 2, in diagnostic evaluations, the system may render (i.e., present) a rendered line corresponding to the gaze ray. The gaze ray rendering may be enabled or disabled. The system may evaluate individual eyes, e.g., for stability, accuracy, pursuit, and attention. The system may evaluate both eyes in combination also for stability, accuracy, pursuit, and attention. The system may employ a standard set of testing parameters. Examples may include the (i) a variance μ(t) of the gaze control point, (ii) a mean location of the gaze control point, (iii) a latency τ(t) of the gaze control point, (iv) a change in the variance Δ_{μ}(t) over time of the gaze control point, (v) a change in the variance Δ_{τ}(t) over time of the gaze control point, (vi) a location s(t) of the gaze control point, (vii) a velocity measure v(t) of the gaze control point, and (viii) an acceleration a(t) of the gaze control point, as discussed herein.

For training and therapeutic applications, the system may also render a rendered line corresponding to the gaze ray to provide biofeedback. The system may enable or disable such feedback between stages of the game/application. The system can perform individual eye training by using only one eye input or measurement for the task. In some embodiments, the system can ask the user to keep both eyes open or to only keep the one eye (subjected to the training) opened. The system can perform training using both eye inputs or measurements.

Table 3 provides examples of visuomotor function analysis that may be performed.

TABLE 3

| | |
|---|---|
| A | # of hits |
| | Accuracy |
| | Time to complete task/game |
| B | Eye movement accuracy and stability during a pre-defined |
| | spatiotemporal window |

Per Table 3, the first type of function analysis includes the number of hits, accuracy, and time to complete the task/game. The second type of function analysis includes the assessed eye movement accuracy and stability during a pre-defined spatiotemporal window.

### Example Applications

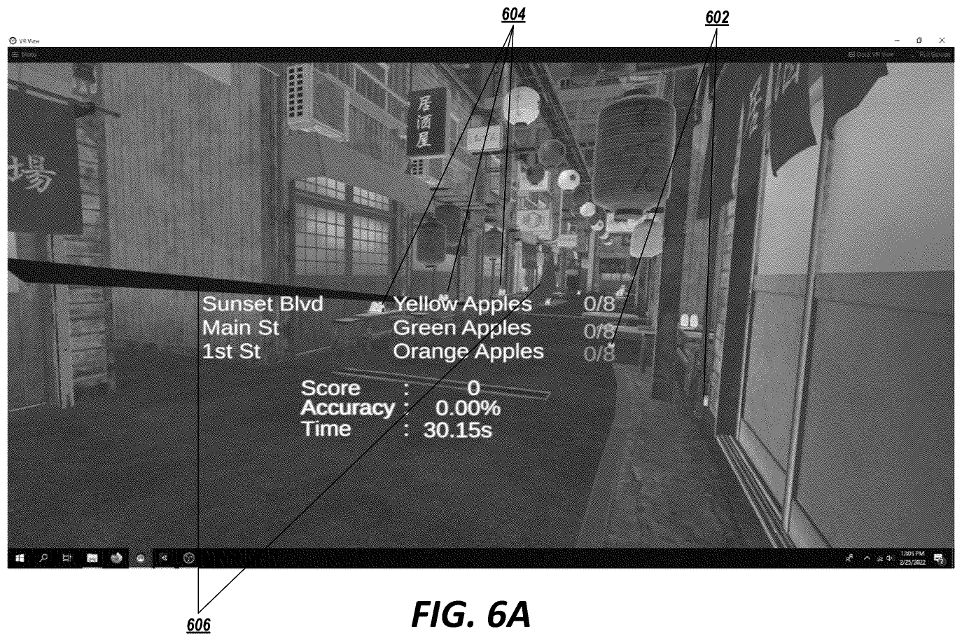

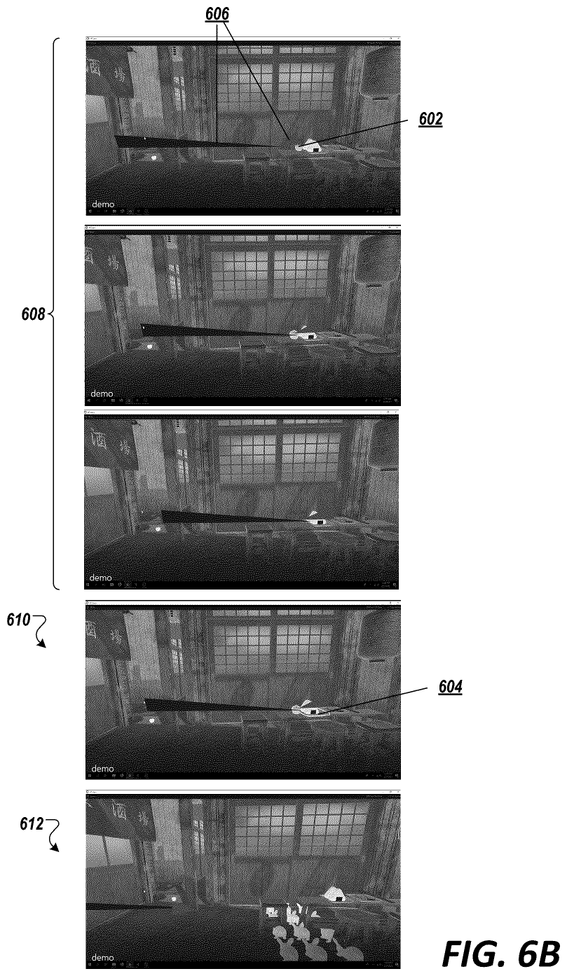

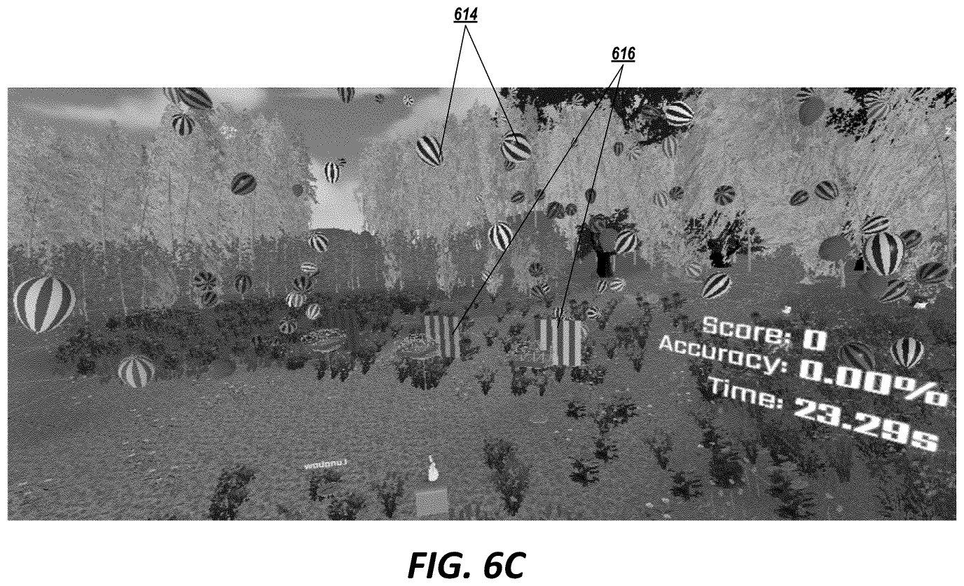

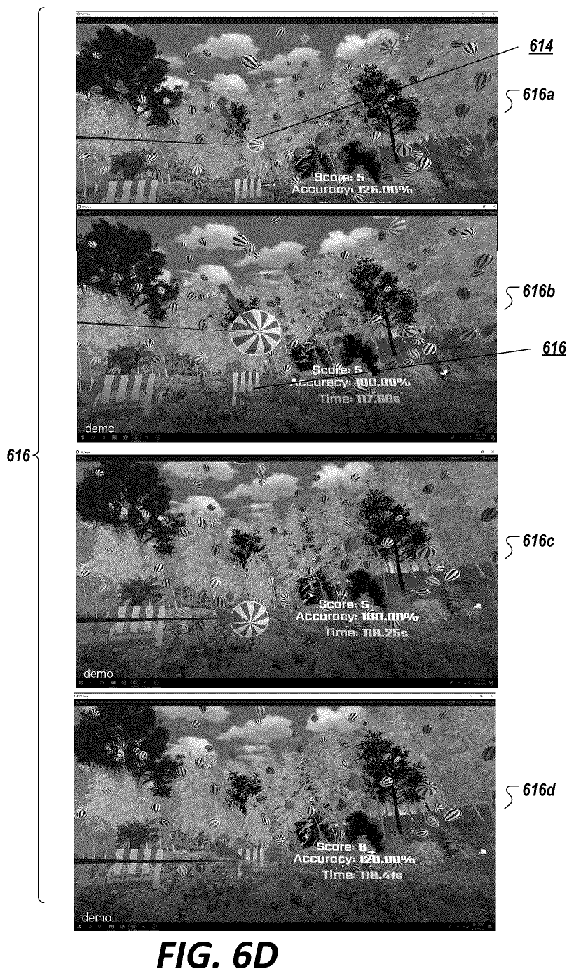

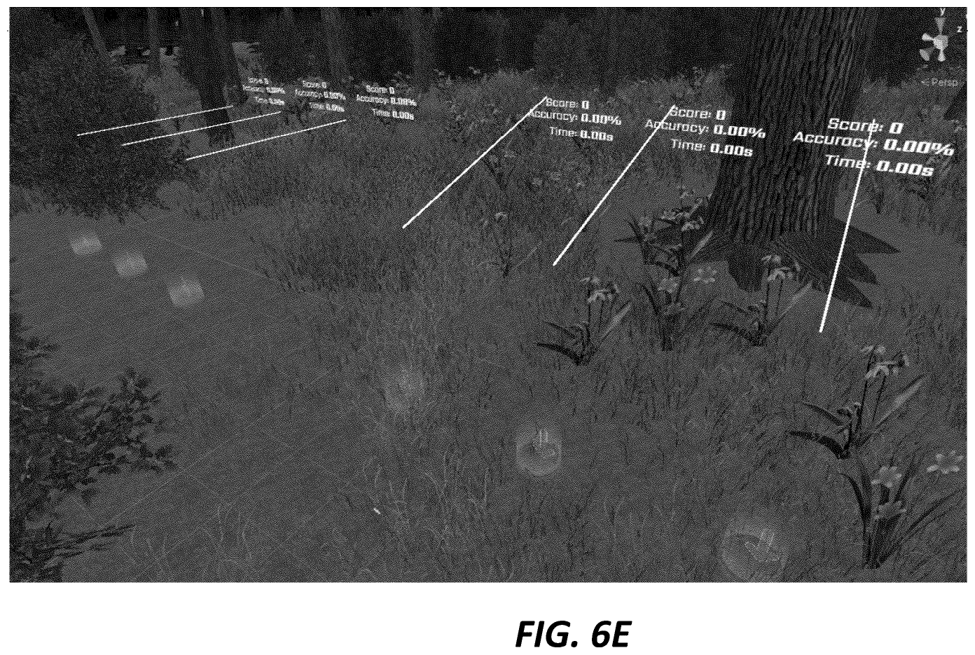

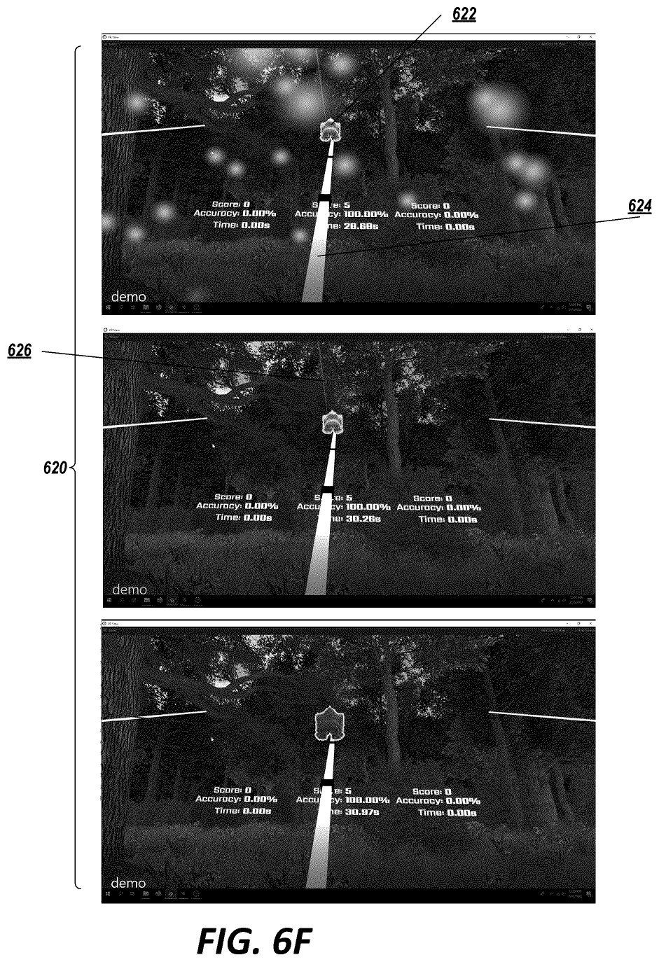

FIGS. 6A-6D show example applications for gaze-associated diagnostics and/or training. Table 4 provides a summary of each game.

TABLE 4

| Game | |
|---|---|
| Name | Description |
| Japanese | This game is similar to the Type “5” application described in |
| Alley | relation to Table 1, in which multiple hidden stationary |
| (FIGS. | objects are concurrently presented for the user, in this case, |
| 6A-6B) | apples, to search for within the scene and then fixate. The user |
| | is able to move within the scene corresponding to a Japanese |
| | alley to search for the object. |
| | The system can train and assess visuomotor functions such as |
| | visual search perception, eye fixation accuracy, eye fixation |
| | stability, and eye saccadic search pattern. The application type |
| | can incorporate application types “1”, “2”, and “3”, e.g., for |
| | different levels of difficulty or stages. |
| Balloons | This game is similar to the Type “4” application described in |
| (FIGS. | relation to Table 1 in which multiple stationary and moving |
| 6C-6D) | objects, in this case, floating balloons, are concurrently |
| | presented for the user to fixate for a period of time to select |
| | and then perform a task with respect to the selected object. In |
| | this application, the user has to fixate on the balloon for a |
| | period of time to select it and then move the balloon in a cage. |
| | The system can train and assess visuomotor functions such as |
| | visual search perception, eye pursuit, eye convergence, and |
| | visual attention. The application type can incorporate |
| | application types “1”, “2”, and “3”, e.g., for different level of |
| | difficulty or stages. |
| Forest | This game is similar to the Type “3” application described in |
| Bugs | relation to Table 1, in which a single object is presented for |
| (FIGS. | the user to fixate, in this case, a bug. The object is |
| 6E-6F) | displaced/moved during the fixation to assess for eye |
| | convergence-saccade and eye convergence pursuit. |

Japanese Alley.

In this game/application, multiple hidden stationary objects are concurrently presented for the user, in this case, apples, to search for within the scene and then fixate. As shown in FIG. 6A, the user is able to move, e.g., via input from the keyboard or teleporting oneself using a controller, within the scene corresponding to a Japanese alley to search for the object. Within a scene, the user is tasked with searching and fixating on a set of colored apples 602 (e.g., red, yellow, green) for 1 second to collect them. The score (e.g., number of apples), accuracy, and time to completion are tracked. An example sequence 608 is shown in FIG. 6B. The system further presents distractors 604 (e.g., sushi and moving objects, e.g., bunnies) during the task, shown in images 610, 612 in FIG. 6B. Collecting distractors other than the colored apples result in penalties to accuracy. The game/application can show the user collecting the objects with gaze rays 606 enabled and then with gaze rays disabled.

Balloons.

In this game/application, multiple stationary and moving objects shown in FIG. 6C as floating balloons 614 are concurrently presented for the user to fixate for a period of time to select and then perform a task of moving the balloon in a cage. The balloons are rendered with stripes, along with stationary distractors 616 that are similarly striped. An example sequence 616 is shown in FIG. 6C. In the sequence 616, the user attempts to fixate (616a) on a floating balloon 614. Upon the fixation performed without interruption for 1 second, the balloon is selected (616b) and is shown enlarged. The user can then move the balloon by slowly moving their fixation to a cage 618 (see 616c). After a period of time of fixating the balloon 614 over the cage 618, the balloon disappears (616d) and a point is registered.

Forest Bugs.

In this game/application, the system presents a set of bugs to be viewed by the user as shown in FIG. 6E. In the sequence 620, the user fixates on a bug 622 on a stick 624 and uses eye convergence (shown per the gaze rays 626) to move the bug to the correct positions along the stick.

### Example Analysis Visualization

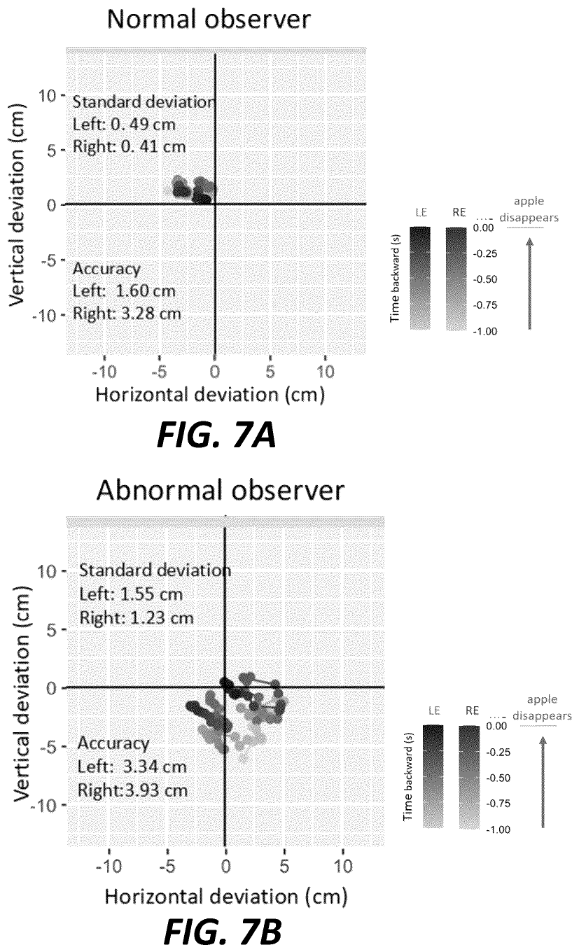

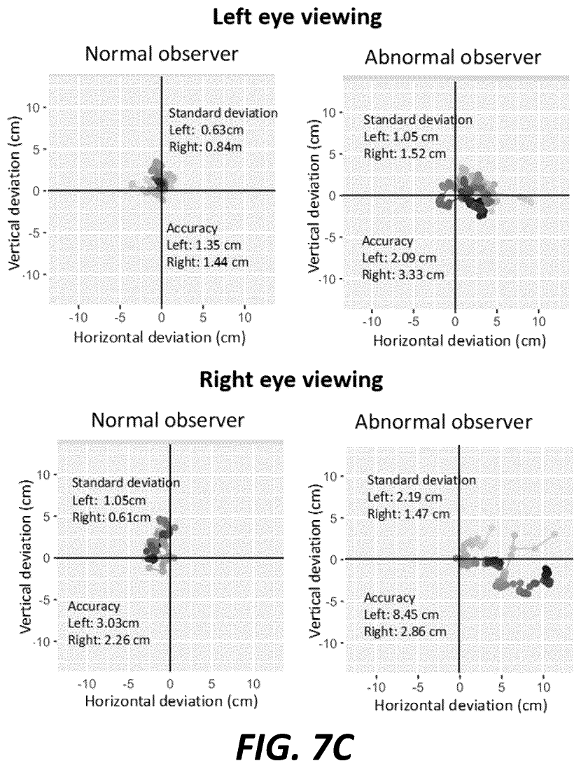

FIGS. 7A-7C show example output analysis visualization for the visuomotor function analysis performed in the diagnostic or training exercise described herein. Specifically, FIGS. 7A and 7B shows example eye fixation data of two subject playing a game (e.g., balloon, forest bug, or Japanese alley) with both eyes viewing on the object or target. FIG. 7A shows data for a normal observer having clinically normal vision, while FIG. 7B shows data for an abnormal observer having a binocular visual disorder (accommodative esotropia). The plots show the data with respect to the horizon and vertical deviations. Indeed, the plot shows statistically significant differences between the two subjects.

Each graph plots the position of the right and left eyes for the 1-second the user had to fixate on the apple to “destroy” it. The intersection of the x and y axes indicates the center of the apple. Eye positioned at any part of the apple is sufficient to “destroy” the apple. It can be observed that the accuracy and standard deviation of eye position are larger in the two eyes of the abnormal observer. This can indicate the abnormal observer has a poorer ability to accurately fixate on the apple and to keep their eye fixation steady over time.

FIG. 7C shows example eye fixation data of two subject playing the game (e.g., balloon, forest bug, or Japanese alley) with either the left or right eye alone viewing the object or target. FIG. 7C shows data for a normal observer having clinically normal vision and data for an abnormal observer having a binocular visual disorder (accommodative esotropia). The plots shows the data with respect to horizon and vertical deviations. Indeed, the plot shows statistically significant differences between the two subjects.

The graphs of FIG. 7C are plotted as in the binocular viewing condition. It can be observed that the accuracy and standard deviation of eye position are smaller in the viewing eye, particularly for the normal observer. This suggests the criterion for fixating on the apple is based on this eye's positions alone. This factor can be used to selectively diagnose and train an eye.

### Experimental Results and Example

A study was conducted to develop gaze control-associated hardware and software that can be used as a part of hand-free user interface input for interactions with a virtual or augmented three-dimensional computing environment in which the gaze control can be used for the assessment of clinical diagnosis of eye movement and fixation problems and therapy.

The study implemented eye-tracking gaze-based control that can augment the capability in an existing off-the-shelf head-mounted display system. For the study, the HTC Vive Pro Eye VR headset was employed running on the Unity game engine. The study employed the eye-tracking gaze-based as hands-free controllers for subjects to interact in the virtual 3D environment, including in various gaming environments. In the study, rather than using a hand-held controller such a mouse pointer or like to interact with a virtual object, the eye-tracking gaze-based was implemented that allowed subjects of the study to pick up the virtual object using eye movements and fixation. That is, the subject conducted various evaluations in the study using only their eyes to remotely pick up a virtual object and move it to a different location in the virtual scene or to interact with the virtual object in the virtual environment.

In the study, the Unity game engine logged each eye's movements and fixation behaviors. The study includes software that interfaced to the Unity game engine drivers to receive eye-tracking information collected by the Unity game engine. To implement the eye-tracking gaze-based control, the study implemented a callback routine that bypassed the Unity engine's tick rate (50 Hz) and accessed the headset hardware's full 120 Hz recording capability. The study then implemented a Python script that could visualize the dynamic eye positions in a 3D spatial plot and implemented application code that was executed in the Unity game engine to replay the movements of the gaze rays from a saved log.

In one test application, the study used the logged eye movements and fixation behaviors in a sensorimotor protocol that required the subject, within the test application, to look at visual objects at various locations in the virtual 3D space generated within the test application. The study observed that eye movement traces and gaze rays generated using the subject's interaction with the visual objects facilitated the identification of the subject's dominant eye. As described herein, identification of eye dominance is an important aspect of improving or training eye-hand coordination, e.g., in athletic and vocational activities.

The study also implemented improved gaze-collision control that calculated a point of binocular convergence in 3D world space using the gaze rays from both eyes to reflect the combined eye movement patterns in 3D space. This improved gaze-collision control replaced the native gaze-collision implementation of the HTC Vive, which simply averaged the gaze rays from each eye and cast this ray into virtual world space until it intersected on an object.

The improved gaze-collision control allows accurate combined eye movement patterns to be employed in 3D space while maintaining the independence of each eye's characteristic pattern of movements that are needed for clinically and diagnostics-relevant measurements. In contrast, averaging the gaze rays as performed by the native gaze collision operation assumes that the two eyes are equally capable of fixating at the object of interest. This is inadequate in assessing the true combined eye movement patterns required for clinically and diagnostics-relevant measurements since a real subject normally has a dominant (preferred) eye that plays a leading role in the eye movements and fixation and which tends to be more accurately fixated on the object.

The study then implemented eye-tracking gaze-based control and improved gaze-collision control in hands-free controller operations in a number of test applications in which the hands-free gaze-control controller is employed in interactive 3D visual scenarios (games) to replace the native hand-controller device.

The hands-free gaze-control controller was employed in a number of test applications comprising games that required subjects to perform a set of tasks as outlined in Table 5.

TABLE 5

| | Task Objectives in Test Applications and Experiments |
|---|---|
| Scenario 1 | Find an object of interest by fixating intently on the object for |
| | a pre-defined or user-selectable time (e.g., 1 or 2-seconds) |
| | (with the right eye, left eye, or both eyes) to cause the object |
| | to vanish |
| Scenario 2 | Fixate on the object of interest, which causes the object to be |
| | held by the gaze, and then move their eyes to place the object |
| | into a basket in 3D |
| Scenario 3 | Move their eyes in the mid-sagittal plane (far-to-near or |
| | near-to-far) to fixate on objects at different depths. |

The experiments developed within the video games were designed to be entertaining for the subject while providing logged data (for each eye) to assess the subject's ability to move/track an object, and the ability of both eyes to work together. It was observed that subjects with superior binocular vision could better control the fixation of their non-dominant eyes, which led to more accurate 3D gaze localization and efficient visual search. The study also observed that showing the visualization of the gaze rays in the test applications, e.g., to serve as a biofeedback mechanism to the subject, helped the subjects to be more accurate in fixating at the object.

Discussion

By furthering the state-of-the-art of VR technology and creating prototype games based on fundamental knowledge of vision science, as explained above, we are able to employ the VR headset system to more precisely measure a subject's ability to control their eye movements and fixation. And since the hardware is an off-the-shelf product that can be used at home and any other setting, it can be made widely available for a broad range of applications. This has clear healthcare implications in that the system could be deployed either as a diagnostic or therapeutic tool. The system, with the gaze rays as biofeedback, could be used diagnostically to reveal the eye movements and fixation deficits of patients with traumatic and neurodegenerative diseases that affect the eye and vision. As a therapeutic tool, it could be used to educate patients about potential difficulties as they navigate the 3D virtual scenes and perform activities of daily living.

Additionally, the system could be used to help one improve performance that relies heavily on eye dominance and location accuracy, such as in aiming and shooting sports. For example, the system could inform an archer whose eye and hand dominances are opposite of how their eyes should be positioned when aiming. Similarly, athletes in other competitive sports whose success depends on milliseconds advantage and superior coordination between the two eyes could improve by learning where to look. Lastly, an added advantage of the VR system is that it can expose the subject to different levels of challenges in the virtual 3D scenes, which are not easy to produce or manipulate in the real 3D environment. For instance, one could create an “entry-level” virtual 3D scene without many distracting objects for a beginner subject tasked to quickly pick up an object of interest with their eye(s) or hand controller. Then, as the subject improves, we can progressively add more distractors into the virtual scene while requiring the subject to perform higher-level perceptual tasks, such as discriminating between the shape, size, or orientation of the object of interest. At the advanced level, the subject could be asked to navigate the virtual scene while picking up the object of interest in the presence of distractors, with points deducted if they veer off the correct path and accidentally pick up the wrong objects.

While velocity, amplitude, and accuracy measures of the eye movements are important metrics, conventional eye-tracking tests do not facilitate the visualization of the eye movement behaviors simultaneously in real-time nor provide instantaneous feedback for the examiner or the subject, which can be crucial for modifying behaviors, e.g., in diagnostic or for therapy of eye-tracking issues.

Furthermore, while people live in a 3D environment and our eyes naturally look at objects both along the frontal and sagittal (depth) planes, we know very little about how our eyes behave in 3D space because most existing technology measures eye movements in 2D. There are benefits to learning about eye movements in 3D and measuring each eye's movements independently. The difference in movements between the two eyes can provide crucial information about their behaviors in 3D.

While the virtual reality environment provides for such opportunities as described herein, existing VR software protocol, for the most part, simply averages the two eyes' movements rather than analyzing the difference. It thus discards or ignores the individual eye's behavioral data.

Furthermore, since the absolute magnitude of the difference between the movements of the two eyes is in the order of minutes of arc, eye-tracking instruments with better spatial and temporal resolutions are required to provide adequate visualization of the resultant 3D eye movement data. Such fine 3D eye movement data can inform clinicians whether a patient with neurological deficits (e.g., from neural degeneration or trauma) also suffers from poor movement coordination between the two eyes, which degrades depth perception. The measured eye movements and fixation behaviors in the 3D visual environment can more fully inform researchers and clinicians of different neural circuitries involved in eye tracking and eye fixation.

Recent technological advances in VR headset systems are making it possible to track eye movements and fixation behaviors in 3D space while wearing the headset, providing a potential utility for measuring eye and head (gaze) movement behaviors in 3D scenes, as well as for visualization of the gaze behaviors simultaneously (online). Though in its infancy, several VR-headset models have incorporated hardware capable of tracking eye movements and fixation while the subject views the virtual scenes in 3D space. In particular, a feature called “gaze rays” can be implemented to allow the subjects to visualize where their eyes are moving and fixating in real time, thus providing them with instantaneous feedback about their ocular-motor behaviors. However, the available software features for analyzing the eye movement patterns are not configured with high-quality controls that are designed to provide a spatial and temporal resolution that would be useful to measure and understand eye movements and fixation behaviors for clinical and evaluative assessment.

### Example Computing System

It should be appreciated that the logical operations described above and in the appendix can be implemented (1) as a sequence of computer-implemented acts or program modules running on a computing system and/or (2) as interconnected machine logic circuits or circuit modules within the computing system. The implementation is a matter of choice dependent on the performance and other requirements of the computing system. Accordingly, the logical operations described herein are referred to variously as state operations, acts, or modules. These operations, acts, and/or modules can be implemented in software, in firmware, in special purpose digital logic, in hardware, and any combination thereof. It should also be appreciated that more or fewer operations can be performed than shown in the figures and described herein. These operations can also be performed in a different order than those described herein.

The computer system is capable of executing the software components described herein for the exemplary method or systems. In an embodiment, the computing device may comprise two or more computers in communication with each other that collaborate to perform a task. For example, but not by way of limitation, an application may be partitioned in such a way as to permit concurrent and/or parallel processing of the instructions of the application. Alternatively, the data processed by the application may be partitioned in such a way as to permit concurrent and/or parallel processing of different portions of a data set by the two or more computers. In an embodiment, virtualization software may be employed by the computing device to provide the functionality of a number of servers that are not directly bound to the number of computers in the computing device. For example, virtualization software may provide twenty virtual servers on four physical computers. In an embodiment, the functionality disclosed above may be provided by executing the application and/or applications in a cloud computing environment. Cloud computing may comprise providing computing services via a network connection using dynamically scalable computing resources. Cloud computing may be supported, at least in part, by virtualization software. A cloud computing environment may be established by an enterprise and/or can be hired on an as-needed basis from a third-party provider. Some cloud computing environments may comprise cloud computing resources owned and operated by the enterprise as well as cloud computing resources hired and/or leased from a third-party provider.

In its most basic configuration, a computing device includes at least one processing unit and system memory. Depending on the exact configuration and type of computing device, system memory may be volatile (such as random-access memory (RAM)), non-volatile (such as read-only memory (ROM), flash memory, etc.), or some combination of the two.

The processing unit may be a standard programmable processor that performs arithmetic and logic operations necessary for the operation of the computing device. While only one processing unit is shown, multiple processors may be present. As used herein, processing unit and processor refers to a physical hardware device that executes encoded instructions for performing functions on inputs and creating outputs, including, for example, but not limited to, microprocessors (MCUs), microcontrollers, graphical processing units (GPUs), and application-specific circuits (ASICs). Thus, while instructions may be discussed as executed by a processor, the instructions may be executed simultaneously, serially, or otherwise executed by one or multiple processors. The computing device may also include a bus or other communication mechanism for communicating information among various components of the computing device.

Computing devices may have additional features/functionality. For example, the computing device may include additional storage such as removable storage and non-removable storage including, but not limited to, magnetic or optical disks or tapes. Computing devices may also contain network connection(s) that allow the device to communicate with other devices, such as over the communication pathways described herein. The network connection(s) may take the form of modems, modem banks, Ethernet cards, universal serial bus (USB) interface cards, serial interfaces, token ring cards, fiber distributed data interface (FDDI) cards, wireless local area network (WLAN) cards, radio transceiver cards such as code division multiple access (CDMA), global system for mobile communications (GSM), long-term evolution (LTE), worldwide interoperability for microwave access (WiMAX), and/or other air interface protocol radio transceiver cards, and other well-known network devices. Computing devices may also have input device(s) such as keyboards, keypads, switches, dials, mice, trackballs, touch screens, voice recognizers, card readers, paper tape readers, or other well-known input devices. Output device(s) such as printers, video monitors, liquid crystal displays (LCDs), touch screen displays, displays, speakers, etc., may also be included. The additional devices may be connected to the bus in order to facilitate the communication of data among the components of the computing device. All these devices are well known in the art and need not be discussed at length here.

The processing unit may be configured to execute program code encoded in tangible, computer-readable media. Tangible, computer-readable media refers to any media that is capable of providing data that causes the computing device (i.e., a machine) to operate in a particular fashion. Various computer-readable media may be utilized to provide instructions to the processing unit for execution. Example tangible, computer-readable media may include but is not limited to volatile media, non-volatile media, removable media, and non-removable media implemented in any method or technology for storage of information such as computer-readable instructions, data structures, program modules, or other data. System memory, removable storage, and non-removable storage are all examples of tangible computer storage media. Example tangible, computer-readable recording media include, but are not limited to, an integrated circuit (e.g., field-programmable gate array or application-specific IC), a hard disk, an optical disk, a magneto-optical disk, a floppy disk, a magnetic tape, a holographic storage medium, a solid-state device, RAM, ROM, electrically erasable program read-only memory (EEPROM), flash memory or other memory technology, CD-ROM, digital versatile disks (DVD) or other optical storage, magnetic cassettes, magnetic tape, magnetic disk storage or other magnetic storage devices.

In light of the above, it should be appreciated that many types of physical transformations take place in the computer architecture in order to store and execute the software components presented herein. It also should be appreciated that the computer architecture may include other types of computing devices, including hand-held computers, embedded computer systems, personal digital assistants, and other types of computing devices known to those skilled in the art.

In an example implementation, the processing unit may execute program code stored in the system memory. For example, the bus may carry data to the system memory, from which the processing unit receives and executes instructions. The data received by the system memory may optionally be stored on the removable storage or the non-removable storage before or after execution by the processing unit.

It should be understood that the various techniques described herein may be implemented in connection with hardware or software or, where appropriate, with a combination thereof. Thus, the methods and apparatuses of the presently disclosed subject matter, or certain aspects or portions thereof, may take the form of program code (i.e., instructions) embodied in tangible media, such as floppy diskettes, CD-ROMs, hard drives, or any other machine-readable storage medium wherein, when the program code is loaded into and executed by a machine, such as a computing device, the machine becomes an apparatus for practicing the presently disclosed subject matter. In the case of program code execution on programmable computers, the computing device generally includes a processor, a storage medium readable by the processor (including volatile and non-volatile memory and/or storage elements), at least one input device, and at least one output device. One or more programs may implement or utilize the processes described in connection with the presently disclosed subject matter, e.g., through the use of an application programming interface (API), reusable controls, or the like. Such programs may be implemented in a high-level procedural or object-oriented programming language to communicate with a computer system. However, the program(s) can be implemented in assembly or machine language, if desired. In any case, the language may be a compiled or interpreted language, and it may be combined with hardware implementations.

Although example embodiments of the present disclosure are explained in some instances in detail herein, it is to be understood that other embodiments are contemplated. Accordingly, it is not intended that the present disclosure be limited in its scope to the details of construction and arrangement of components set forth in the following description or illustrated in the drawings. The present disclosure is capable of other embodiments and of being practiced or carried out in various ways.

It must also be noted that, as used in the specification and the appended claims, the singular forms “a,” “an,” and “the” include plural referents unless the context clearly dictates otherwise. Ranges may be expressed herein as from “about” or “approximately” one particular value and/or to “about” or “approximately” another particular value. When such a range is expressed, other exemplary embodiments include from the one particular value and/or to the other particular value.

By “comprising” or “containing” or “including” is meant that at least the name compound, element, particle, or method step is present in the composition or article or method, but does not exclude the presence of other compounds, materials, particles, method steps, even if the other such compounds, material, particles, method steps have the same function as what is named.

In describing example embodiments, terminology will be resorted to for the sake of clarity. It is intended that each term contemplates its broadest meaning as understood by those skilled in the art and includes all technical equivalents that operate in a similar manner to accomplish a similar purpose. It is also to be understood that the mention of one or more steps of a method does not preclude the presence of additional method steps or intervening method steps between those steps expressly identified. Steps of a method may be performed in a different order than those described herein without departing from the scope of the present disclosure. Similarly, it is also to be understood that the mention of one or more components in a device or system does not preclude the presence of additional components or intervening components between those components expressly identified.

The term “about,” as used herein, means approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 10%. In one aspect, the term “about” means plus or minus 10% of the numerical value of the number with which it is being used. Therefore, about 50% means in the range of 45%-55%. Numerical ranges recited herein by endpoints include all numbers and fractions subsumed within that range (e.g., 1 to 5 includes 1, 1.5, 2, 2.75, 3, 3.90, 4, 4.24, and 5).

Similarly, numerical ranges recited herein by endpoints include subranges subsumed within that range (e.g., 1 to 5 includes 1-1.5, 1.5-2, 2-2.75, 2.75-3, 3-3.90, 3.90-4, 4-4.24, 4.24-5, 2-5, 3-5, 1-4, and 2-4). It is also to be understood that all numbers and fractions thereof are presumed to be modified by the term “about.”

The following patents, applications, and publications as listed below and throughout this document, are hereby incorporated by reference in their entirety herein.
[1] Bastani, Behnam, et al. “Foveated pipeline for AR/VR head-mounted displays.” Information Display 33.6 (2017): 14-35.
[2] U.S. Ser. No. 10/157,313B1.
[3] U.S. Ser. No. 10/401,953B2.
[4] U.S. Ser. No. 10/620,700B2.
[5] U.S. Ser. No. 11/217,033B1.
[6] US20200225743A1.

What is claimed is:

1. A system comprising:
a virtual-reality headset or augmented-reality system configured to measure eye-associated positions and gaze-associated direction; and
an analysis system having a processor and a memory having instructions stored thereon, wherein execution of the instructions by the processor causes the processor to:
generate a rendered scene and/or object in the virtual-reality headset or augmented-reality system;
receive the measured eye-associated positions and measured gaze-associated directions of a user viewing the rendered scene and/or object;
determine a gaze control point from the measured eye-associated positions and measured gaze-associated directions, wherein the gaze control point is a binocular convergence in the rendered scene and/or on the object using the measured gaze-associated directions from both eyes to reflect a combined eye movement pattern.

2. The system of claim 1, wherein execution of the instructions by the processor further cause the processor to:
generate a rendered gaze ray associated with the binocular convergence.

3. The system of claim 1, wherein execution of the instructions by the processor further cause the processor to:
determine one or more statistical parameters, or associated values, from the determined gaze control point.

4. The system of claim 3, wherein the determined one or more statistical parameters includes at least one of:
(i) a variance measure of the gaze control point,
(ii) a mean location of the gaze control point,
(iii) a latency measure of the gaze control point,
(iv) a change in the variance of the gaze control point over time,
(v) a change in the latency of the gaze control point over time,
(vi) an instantaneous location of the gaze control point,
(vii) an instantaneous velocity measure of the gaze control point, and
(viii) an instantaneous acceleration measure of the gaze control point.

5. The system of claim 1, wherein the determined gaze control point, or statistics derived therefrom, are subsequently employed to assess at least one of: eye dominance measure, vestibular measure, visual fixation measure, optokinetic measure, smooth pursuit measure, nystagmus quick phase measure, saccades measure, vergence measure, or a combination thereof.

6. The system of claim 1, wherein the determined gaze control point, or statistics derived therefrom, are subsequently employed in a therapy to address an eye-tracking problem or a disease.

7. A system comprising:
an analysis system having a processor and a memory having instructions stored thereon, wherein execution of the instructions by the processor causes the processor to:
generate a rendered scene and/or object in a virtual-reality headset or augmented-reality system, wherein the virtual-reality headset or augmented-reality system configured to measure eye-associated positions and gaze-associated direction;
receive the measured eye-associated positions and measured gaze-associated directions of a user viewing the rendered scene and/or object;
determine a gaze control point from the measured eye-associated positions and measured gaze-associated directions, wherein the gaze control point is a binocular convergence in the rendered scene and/or on the object using the measured gaze-associated directions from both eyes to reflect a combined eye movement pattern.

8. A system of claim 7, wherein the system is the virtual-reality headset, the visual-reality headset comprising a processor; and a memory having instructions stored thereon, wherein execution of the instructions by the processor causes the processor to (i) display a rendered scene and/or object, (ii) measure eye-associated positions of a user wearing the virtual-reality headset, (iii) measure gaze-associated directions of the user; and (iv) determine a gaze control point from the measured eye-associated positions and measured gaze-associated directions, wherein the gaze control point is a binocular convergence in the rendered scene and/or on the object using the measured gaze associated directions from both eyes to reflect a combined eye movement pattern.

9. The system of claim 7, wherein the determined gaze control point, or statistics derived therefrom, are subsequently employed to assess at least one of: eye dominance measure, vestibular measure, visual fixation measure, optokinetic measure, smooth pursuit measure, nystagmus quick phase measure, saccades measure, vergence measure, or a combination thereof.

10. The system of claim 7, wherein the determined gaze control point, or statistics derived therefrom, are subsequently employed in a therapy to address an eye-tracking problem or a disease.

11. The system of claim 7, wherein the determined one or more statistical parameters includes at least one of:
(i) a variance measure of the gaze control point,
(ii) a mean location of the gaze control point,
(iii) a latency measure of the gaze control point,
(iv) a change in the variance of the gaze control point over time,
(v) a change in the latency of the gaze control point over time,
(vi) an instantaneous location of the gaze control point,
(vii) an instantaneous velocity measure of the gaze control point, and
(viii) an instantaneous acceleration measure of the gaze control point.

12. The system of claim 11, wherein the determined gaze control point, or statistics derived therefrom, are subsequently employed to assess at least one of: eye dominance measure, vestibular measure, visual fixation measure, optokinetic measure, smooth pursuit measure, nystagmus quick phase measure, saccades measure, vergence measure, or a combination thereof.

13. A non-transitory computer-readable medium having instructions stored thereon, wherein execution of the instructions by a processor causes the processor to:
generate a rendered scene and/or object in the virtual-reality headset or augmented-reality system;
receive, from a virtual-reality headset or augmented-reality system, measured eye associated positions and measured gaze-associated directions of a user viewing the rendered scene and/or object;
determine a gaze control point from the measured eye associated positions and measured gaze-associated directions, wherein the gaze control point is a binocular convergence in the rendered scene and/or on the object using the measured gaze associated directions from both eyes to reflect a combined eye movement pattern.

14. The non-transitory computer-readable medium of claim 13, wherein the determined gaze control point, or statistics derived therefrom, are subsequently employed to assess at least one of: eye dominance measure, vestibular measure, visual fixation measure, optokinetic measure, smooth pursuit measure, nystagmus quick phase measure, saccades measure, vergence measure, or a combination thereof.

15. The non-transitory computer-readable medium of claim 13, wherein the determined gaze control point, or statistics derived therefrom, are subsequently employed in a therapy to address an eye-tracking problem or a disease.

16. The non-transitory computer-readable medium of claim 15, wherein the instructions when executed by the processor further cause the processor to generate a rendered gaze ray associated with the binocular convergence.

17. The non-transitory computer-readable medium of claim 15 wherein execution of the instructions by the processor further cause the processor to determine one or more statistical parameters, or associated values, from the determined gaze control point.

18. The non-transitory computer-readable medium of claim 17, wherein the determined one or more statistical parameters includes at least one of:
(i) a variance measure of the gaze control point,
(ii) a mean location of the gaze control point,
(iii) a latency measure of the gaze control point,
(iv) a change in the variance of the gaze control point over time,
(v) a change in the latency of the gaze control point over time,
(vi) an instantaneous location of the gaze control point,
(vii) an instantaneous velocity measure of the gaze control point, and
(viii) an instantaneous acceleration measure of the gaze control point.

19. The non-transitory computer-readable medium of claim 13, wherein the determined gaze control point, or statistics derived therefrom, are subsequently employed to assess at least one of: eye dominance measure, vestibular measure, visual fixation measure, optokinetic measure, smooth pursuit measure, nystagmus quick phase measure, saccades measure, vergence measure, or a combination thereof.

20. The non-transitory computer-readable medium of claim 13, wherein the determined gaze control point, or statistics derived therefrom, are subsequently employed in a therapy to address an eye-tracking problem or a disease.
